(19) United States
(12) Patent Application Publication
(10) Pub. No.: US 2025/0073379 A1
(43) Pub. Date: Mar. 6, 2025

(54) BREAST PUMP WEARABLE IN BRA

(71) Applicant: JIAN CHEN, XINYU CITY (CN)

(72) Inventors: JIAN CHEN, XINYU CITY (CN); FAGEN XIE, XINYU CITY (CN)

(21) Appl. No.: 18/676,452

(22) Filed: May 28, 2024

(30) Foreign Application Priority Data
Aug. 29, 2023 (CN) 202322333954.1

(51) Int. Cl. A61M 1/06

(52) U.S. Cl. CPC A61M 1/06

(57) ABSTRACT

A breast pump wearable in a bra includes a main body containing battery, air pump, circuit control device and diaphragm. The breastshield includes a breast accommodating chamber, a nipple accommodating chamber and a diaphragm installation chamber. A mouth of the diaphragm installation chamber has a chamber cover having a first pipe joint communicating with an air-side vacuum chamber. The diaphragm is sealingly combined with the mouth of the diaphragm installation chamber. A milk-side vacuum chamber is formed between the diaphragm and the diaphragm installation chamber. The air-side vacuum chamber is formed between the diaphragm and the chamber cover. The diaphragm installation chamber has an air hole communicating with the milk-side vacuum chamber and the nipple accommodating chamber. The breastshield installation chamber has a second pipe joint connected with an air path of the air pump. The second pipe joint is detachably connected to the first pipe joint.

### FIELD OF THE DISCLOSURE

The present disclosure relates to the technical field of breast pumps, and more particularly to a breast pump wearable in a bra.

### BACKGROUND OF THE DISCLOSURE

A breast pump is a device that extracts milk from the breasts of a nursing woman. A breast pump wearable in a bra is an all-in-one breast pump that integrates a suction generating device, a milk collection device and a breast shield, as a wearable breast pump in an independent bra disclosed in CN216798371 U, for example. The breast pump wearable in a bra can be used in the bra as a whole, and the user does not need to take off the clothes or enter a specific position when pumping breast milk, so the breast pump wearable in a bra has the advantage of being convenient to use in public places such as offices.

In the above-mentioned patent document, the holding part of the diaphragm is arranged on the breastshield, and the holding part and the lower part of the diaphragm jointly form a milk-side vacuum chamber, and an air-side vacuum chamber is defined by a concave part arranged in the housing of the breast pump and a upper part of the diaphragm. When working, the air pump drives the diaphragm to create a negative pressure inside the breastshield to simulate the suction generated when a nursing woman is feeding a child. Since the diaphragm in the above-mentioned patent document is jointly fixed by the breastshield and the breast pump housing, the fixation of the diaphragm is released when the breastshield is disassembled. Therefore, when the breastshield is put back again, an aligning operation of the diaphragm and the holding part of the diaphragm and an aligning operation of the diaphragm and the concave part on the housing of the breast pump are required. The breastshield needs to be disassembled and cleaned frequently because it contains a milk channel, which makes the cleaning and maintenance operations of the breast pump more complicated.

### SUMMARY OF THE DISCLOSURE

The purpose of the present disclosure is to provide a breast pump wearable in a bra to simplify an installation operation of the breastshield and make the cleaning and maintenance of the breastshield more convenient.

In order to solve the above-mentioned problems, the technical solution adopted by the present disclosure is as follows.

A breast pump wearable in a bra is provided, which includes: a main body including a battery, an air pump and a circuit control device; a milk collection container detachably connected to the main body; a breastshield detachably connected to the main body and in communication with the milk collection container; and a diaphragm. The breastshield has a breast accommodating chamber, a nipple accommodating chamber and a diaphragm installation chamber. A mouth of the diaphragm installation chamber is provided with a chamber cover. The diaphragm is sealingly combined with the mouth of the diaphragm installation chamber. A milk-side vacuum chamber is formed between the diaphragm and the diaphragm installation chamber. An air-side vacuum chamber is formed between the diaphragm and the chamber cover. The chamber cover is provided with a first pipe joint in spatial communication with the air-side vacuum chamber. The diaphragm installation chamber is provided with an air hole in spatial communication with the milk-side vacuum chamber and the nipple accommodating chamber. The breastshield installation chamber of the main body is provided with a second pipe joint connected to an air path of the air pump. The second pipe joint is detachably connected to the first pipe joint.

The above-mentioned breastshield has the diaphragm installation chamber, the mouth of the diaphragm installation chamber is provided with the chamber cover, the diaphragm is sealingly combined with the mouth of the diaphragm installation chamber, the milk-side vacuum chamber is formed between the diaphragm and the diaphragm installation chamber, and the air-side vacuum chamber is formed between the diaphragm and the chamber cover, which makes a fixation of the diaphragm, a formation of the milk-side vacuum chamber and a formation of the air-side vacuum chamber all independent of the main body, and since all the aforementioned components are integrated on the breastshield, there is no need to align the diaphragm when mounting the breastshield to the main body. By virtue of that the chamber cover is provided with the first pipe joint in communication with the air-side vacuum chamber, the breastshield installation chamber of the main body is provided with the second pipe joint in communication with the air path of the air pump, and the second pipe joint is detachably connected to the first pipe joint, when the breastshield is mounted to the main body, the air path connection of the diaphragm can be completed by inserting the first pipe joint with the second pipe joint, thereby making the cleaning and maintenance of the breastshield more convenient.

Preferably, the breastshield has a base made of a rigid material and a cover body made of a soft rubber. The diaphragm installation chamber is arranged on the base, the breast accommodating chamber and the nipple accommodating chamber are correspondingly arranged on the cover body, the base is connected to a periphery of the nipple accommodating chamber, and a milk channel from the nipple accommodating chamber to the milk collection container is arranged inside the base.

The above-mentioned breastshield includes the base made of a rigid material and the cover body made of a soft rubber, and the breast accommodating chamber and the nipple accommodating chamber are arranged in the cover body, so that the user feels more comfortable when wearing the breastshield, and an airtightness of the joints between the breastshield and the breast becomes higher. The diaphragm installation chamber is arranged on the base, and the base is made of the rigid material, so that a relative position between the diaphragm installation chamber and the main body is fixedly maintained, and the vibration of the diaphragm installation chamber due to the deformation of the cover body of the breastshield during use is avoided.

Preferably, the base of the breastshield is provided with a third pipe joint for outputting milk, the main body is provided with a one-way valve corresponding to the third pipe joint, and the third pipe joint is detachably connected to an inlet port of the one-way valve.

The above-mentioned third pipe joint not only serves as a milk channel, but also serves as a connection and positioning mechanism between the breastshield and the main body, so that the connection mechanism between the breastshield and the main body can be simplified. In addition, the arrangement of the one-way valve can avoid the backflow of milk in the milk collection container.

Preferably, the base of the breastshield has a columnar main body, the third pipe joint extends from a side of the columnar main body, the breastshield installation chamber of the main body has a columnar connection chamber, an installation hole for the one-way valve is provided on a side of the columnar connection chamber, and the columnar main body is detachably connected to the columnar connection chamber.

The columnar main body, the columnar connection chamber, the third pipe joint and the inlet port of the one-way valve together form the connection and positioning mechanism between the breastshield and the main body. When the breastshield is installed on the main body, the third pipe joint and the inlet port of the one-way valve are aligned and inserted, and the columnar main body is pushed into the columnar connection chamber in the main body to complete the installation. When the breastshield is disassembled from the main body, it only needs to pull the breastshield outward from the main body, so the disassembly and installation of the breastshield becomes more convenient.

Preferably, one side of the columnar main body opposite to the third pipe joint is provided with a convex portion configured for user's fingers to move, and the convex portion extends to a mouth edge of the breastshield.

The arrangement of the convex portion makes it easy for the user's fingers to pull the movable breastshield outward toward the main body, so that the disassembly operation of the breastshield becomes easier.

Preferably, the main body has a first end for connecting to the milk collection container and a second end facing away from the user's breast, and the first end and the second end are adjacent to each other. The milk collection container has a third end fitting with the first end, and a fourth end forming an outer end of the breast pump together with the second end. The third end is provided with at least one convex hook extending toward the first end, and the first end is provided with a hole buckle fitting with the at least one convex hook. The convex hook has a rod portion and a reverse hook portion at an end of the rod portion. The hole buckle has a hole through which the rod portion passes and an edge fittingly engaged to the reverse hook portion. The second end is provided with a rotary lock configured to lock the fourth end on the second end.

In the above solution, when combining the milk collection container with the main body, the convex hook is inserted into the hole buckle, and then the rotary lock is rotated by 90 degrees. When the milk collection container is disassembled from the main body, the rotary lock is rotated by 90 degrees in the opposite direction, and then the convex hook is released from the hole buckle. The combination and disassembly of the milk collection container and the main body are very convenient.

Preferably, the third end has an insertion hole for inserting the one-way valve into the milk collection container, and the one-way valve is fixed to the first end of the main body.

The above solution makes it possible to pour out the milk in the milk collection container through the insertion hole after the milk collection container is disassembled from the main body.

Preferably, a milk extraction hole is provided at a position of the milk collection container away from the third end, and the milk extraction hole is provided with a sealing cover.

In the above solution, the milk extraction hole is provided at the position of the milk collection container away from the third end, and the milk extraction hole is provided with the sealing cover. When the sealing cover is opened, the milk in the milk collection container can flow out from the milk extraction hole under the action of gravity, so that the milk can also be taken out without disassembling the milk collection container, which further facilitates the operation of extracting milk from the breast pump.

Preferably, the circuit control device includes a Bluetooth® module configured to receive an external control signal.

In the above solution, the circuit control device is provided with the Bluetooth® module. By installing an application program in a smart phone equipped with the Bluetooth® module, the smart phone can be used to remotely control the breast pump without operating the buttons on the breast pump, so that the control and operation of the breast pump becomes more convenient.

Preferably, the breast pump includes a dedicated remote controller.

Compared with the prior art, the present disclosure has at least the following beneficial effects.

The breastshield of the present disclosure has the diaphragm installation chamber, the mouth of the diaphragm installation chamber is provided with the chamber cover, the diaphragm is sealingly combined with the mouth of the diaphragm installation chamber, the milk-side vacuum chamber is formed between the diaphragm and the diaphragm installation chamber, and the air-side vacuum chamber is formed between the diaphragm and the chamber cover, which makes the fixation of the diaphragm, the formation of the milk-side vacuum chamber and the formation of the air-side vacuum chamber all independent of the main body, and since all the aforementioned components are integrated on the breastshield, there is no need to align the diaphragm when arranging the breastshield to the main body. By virtue of that the chamber cover is provided with the first pipe joint in communication with the air-side vacuum chamber, the breastshield installation chamber of the main body is provided with the second pipe joint in communication with the air path of the air pump, and the second pipe joint is detachably connected to the first pipe joint, when the breastshield is mounted to the main body, the air path connection of the diaphragm can be completed by inserting the first pipe joint with the second pipe joint, thereby making the cleaning and maintenance of the breastshield more convenient.

### BRIEF DESCRIPTION OF THE DRAWINGS

FIG. 1 is a perspective view of a breast pump according to an embodiment;

FIG. 2 is another perspective view of the breast pump according to an embodiment;

FIG. 3 is a schematic diagram of an air pump, a battery and a circuit control device in a main body;

FIG. 4 is a schematic diagram of the breastshield being disassembled;

FIG. 5 is a schematic diagram of the breastshield;

FIG. 6 is a sectional view of the breastshield;

FIG. 7 is an enlarged view of part A in FIG. 6;

FIG. 8 is a sectional view of the breast pump according to an embodiment;

FIG. 9 is a schematic diagram of a milk collection container according to an embodiment;

FIG. 10 is a schematic diagram of the main body according to an embodiment;

FIG. 11 is another sectional view of the breast pump according to an embodiment; and

FIG. 12 is an enlarged view of part B in FIG. 11.

Reference numeral: 100, main body; 200, milk collection container; 300, breastshield; 101, control button; 102, display unit; 103, air pump; 104, circuit control device; 105, battery; 106, first chamber; 107, second chamber (column connection chamber); 108, third chamber; 109, second pipe joint; 110, one-way valve; 111, second end; 112, rotary lock; 113, first end; 114, hole buckle; 1142, edge; 201, sealing cover; 202, fourth end; 203, insertion hole; 204, third end; 302, cover body; 303, diaphragm installation chamber; 3031, inner ring wall; 304, first pipe joint; 305, chamber cover; 3051, convex ring; 306, air-side vacuum chamber; 307, diaphragm; 3071, fixed ring; 308, milk-side vacuum chamber; 309, breast accommodating chamber; 310, nipple accommodating chamber; 311, columnar main body; 312, third pipe joint; 313, milk channel; and 314, air hole.

### DETAILED DESCRIPTION OF THE EXEMPLARY EMBODIMENTS

The present disclosure will be further described in conjunction with the following drawings and embodiments.

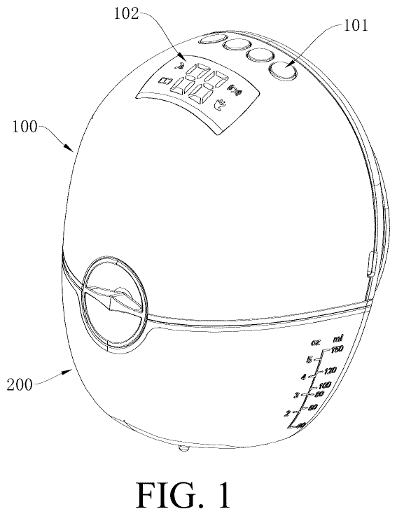

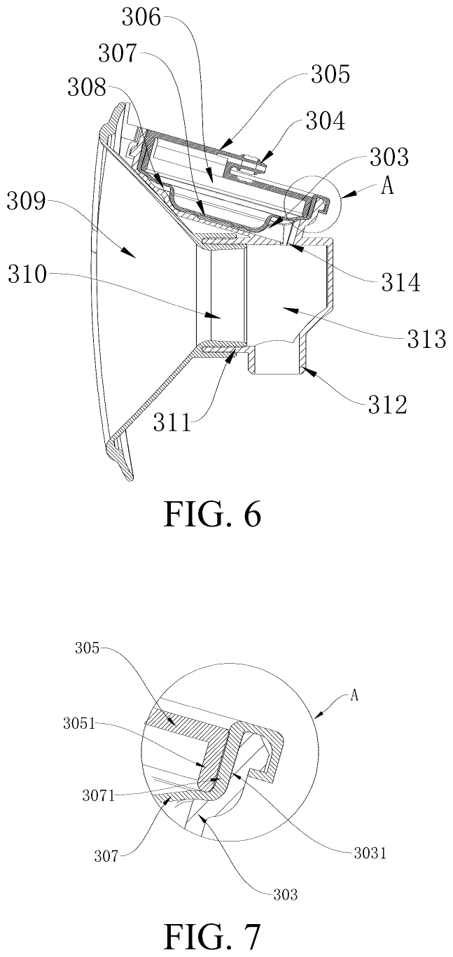

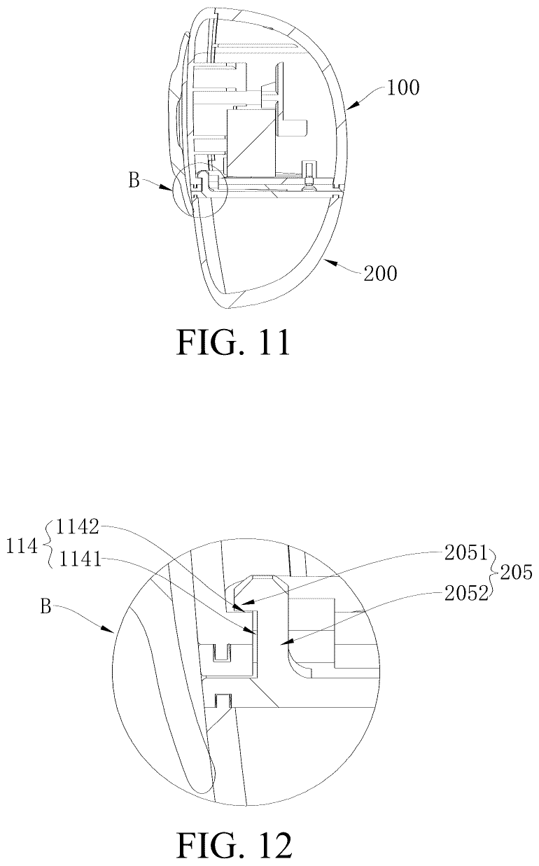

Reference is made to FIGS. 1-12, in which the breast pump in the present embodiment is a breast pump wearable in a bra, and the breast pump includes: a main body 100, a milk collection container 200, a breastshield 300, and a diaphragm 307. The milk collection container 200 is detachably connected to the main body 100, and the breastshield 300 is detachably connected to the main body 100 and in communication with the milk collection container 200.

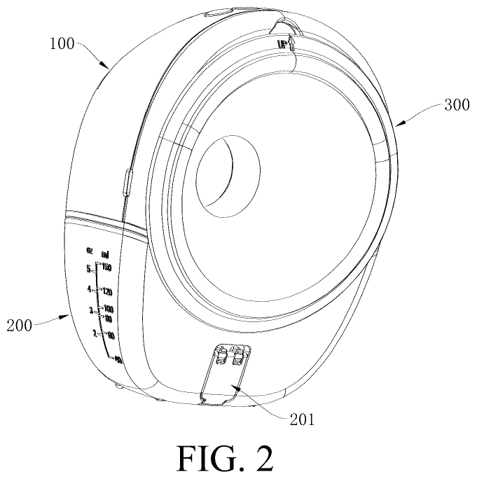

As shown in FIGS. 1 and 2, it is preferable to set a side of the breast pump opposite to the breastshield 300 as a convex arc shape, so that the breast pump can better fit an inner surface of the bra.

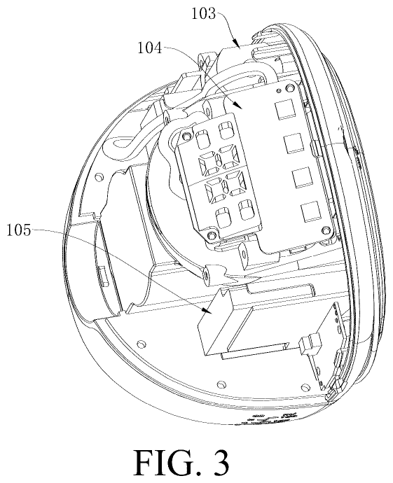

As shown in FIG. 3, a battery 105, an air pump 103 and a circuit control device 104 are disposed inside the main body 100. The battery 105 is used to provide electric energy to electric components such as the circuit control device 104 and the air pump 103, and is preferably a secondary battery. The air pump 103 is used to drive the diaphragm 307 to create a negative pressure inside the breastshield 300 to simulate a suction generated when a nursing woman is feeding a child. The circuit control device 104 can be used to control a charge and discharge of the battery 105, an operation of the air pump 103, an adjustment of the suction force, and so on.

Preferably, as shown in FIG. 1, a control button 101 and a display unit 102 are provided on a surface of the main body 100, and the control button 101 is electrically connected to the circuit control device 104 to realize a control of the breast pump. Preferably, the control button 101 includes one or more of a power switch, a suction adjustment button, and a pause button. Preferably, the control button 101 is arranged on the side of the breast pump opposite to the milk collection container 200, so that a user can operate the control button 101 conveniently.

Preferably, the circuit control device 104 is provided with a wireless communication module, and the wireless communication module can communicate with an external device. As one embodiment, the wireless communication module adopts a Bluetooth© module. In such an embodiment, a smart phone can be used to control the breast pump. Specifically, the application program of the breast pump is installed into the application program on the smart phone, and the breast pump is remotely controlled through the smart phone. Since there is no need to bend the arm to the chest for operation, this method has the advantage of more convenient operation compared with the control button 101 on the breast pump.

As another embodiment, the breast pump further includes a dedicated remote controller. In this manner, the breast pump can be controlled by the dedicated remote controller. The dedicated remote controller can be an infrared remote controller using infrared light waves as a communication medium, or a remote controller using radio as a communication medium.

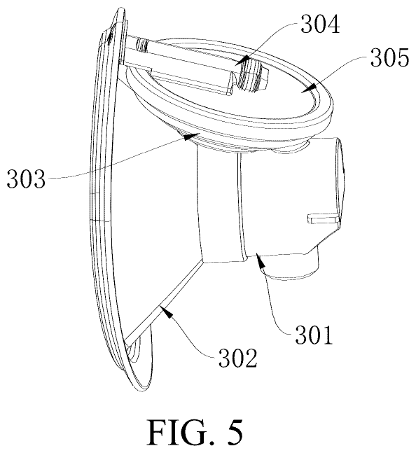

Reference is made to FIGS. 5-7. The breastshield 300 has a breast accommodating chamber 309, a nipple accommodating chamber 310 and a diaphragm installation chamber 303.

Preferably, the breastshield 300 has a base 301 made of a rigid material and a cover body 302 made of a soft rubber material. The diaphragm installation chamber 303 is arranged on the base 301, and the breast accommodating chamber 309 and the nipple accommodating chamber 310 are correspondingly arranged on the cover body 302. The base 301 is connected to a periphery of the nipple accommodating chamber 310, and a milk channel 313 is provided inside the base 301 from the nipple accommodating chamber 310 to the milk collection container 200.

The soft rubber material, for example, can be silica gel, rubber, etc. and the cover body 302 is made of the soft rubber material, which makes the user feel more comfortable when wearing the breast pump wearable in a bra provided by the present disclosure, and makes a joint between the breastshield 300 and the breast more airtight. In addition, the negative pressure generated by the diaphragm 307 is not easy to leak out from the joint between the breastshield 300 and the breast.

The rigid material, for example, can be hard plastic, stainless steel, etc. The base 301 is made of the rigid material so that the relative position between the diaphragm installation chamber 303 and the main body 100 is fixedly maintained, and the vibration of the diaphragm installation chamber 303 due to the deformation of the cover body 302 of the breastshield 300 during use is avoided.

In addition, the base 301 is detachably connected to the cover body 302 to form the breastshield 300, so that it is beneficial to disassemble the cover body 302 to clean the cover body 302 separately.

It should be understood that the breastshield 300 may also be entirely made of the rigid material or the soft rubber material.

A mouth of the diaphragm installation chamber 303 is provided with a chamber cover 305, and the diaphragm 307 is sealedly combined with the mouth of the diaphragm installation chamber 303. A milk-side vacuum chamber 308 is formed between the diaphragm 307 and the diaphragm installation chamber 303. An air-side vacuum chamber 306 is formed between the diaphragm 307 and the chamber cover 305.

Specifically, a fixed ring 3071 can be provided on a periphery of the diaphragm 307, and the fixed ring 3071 can be attached to an inner ring wall 3031 of the diaphragm installation chamber 303. A convex ring 3051 can be arranged on a periphery of the chamber cover 305. The convex ring 3051 of the chamber cover 305 cooperates with the inner ring wall 3031 of the diaphragm installation chamber 303 to squeeze and fix the fixed ring 3071 of the diaphragm 307, and seal the diaphragm 307 to the mouth of the diaphragm installation chamber 303 (as shown in FIG. 7).

An edge of the diaphragm 307 can extend to an outer ring wall of the diaphragm installation chamber 303, a convex ring is arranged on the periphery of the chamber cover 305, and the convex ring of the chamber cover 305 cooperates with the outer ring wall of the diaphragm installation chamber 303 to squeeze and fix the part of the diaphragm 307 extending to the outer ring wall, and sealedly combine the diaphragm 307 with the mouth of the diaphragm installation chamber 303.

The diaphragm 307 can also be fixed by glue.

The chamber cover 305 is preferably made of the rigid material, such as hard plastic, stainless steel, etc.

The diaphragm installation chamber 303 is provided with an air hole 314 in spatial communication with the milk-side vacuum chamber 308 and the nipple accommodating chamber 310.

The diaphragm 307 is made of the soft rubber material, and can be retracted into the air-side vacuum chamber 306 under an action of the air pressure of the air pump 103. When the diaphragm 307 retracts toward the air-side vacuum chamber 306, one negative pressure is generated in the milk-side vacuum chamber 308, so that another negative pressure is generated in the nipple accommodating chamber 310 to simulate the suction generated when a nursing woman is feeding a child at the nipple. After the diaphragm 307 is reset, the negative pressure disappears.

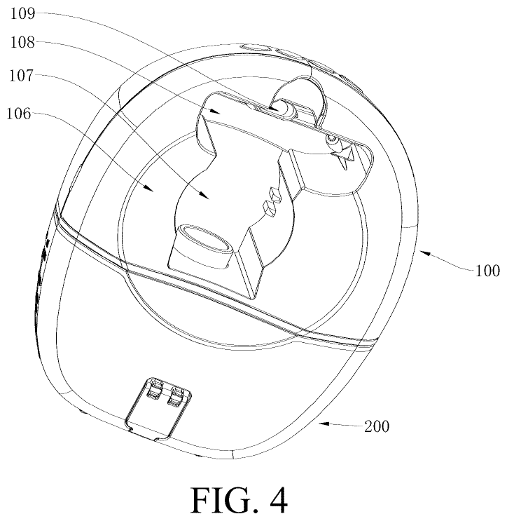

Reference is made to FIGS. 4-8, in which the chamber cover 305 is provided with a first pipe joint 304 in spatial communication with the air-side vacuum chamber 306, and the breastshield installation chamber of the main body 100 is provided with a second pipe joint 109 connected to an air path of the air pump 103. The second pipe joint 109 is detachably connected to the first pipe joint 304. The detachable connection may be that the first pipe joint 304 is inserted into the second pipe joint 109, or the second pipe joint 109 is inserted into the first pipe joint 304.

The breastshield 300 has the diaphragm installation chamber 303. The mouth of the diaphragm installation chamber 303 is provided with the chamber cover 305, the diaphragm 307 is sealingly combined with the mouth of the diaphragm installation chamber 303, the milk-side vacuum chamber 308 is formed between the diaphragm 307 and the diaphragm installation chamber 303, and the air-side vacuum chamber 306 is formed between the diaphragm 307 and the chamber cover 305, which makes the fixation of the diaphragm 307, the formation of the milk-side vacuum chamber 308 and the formation of the air-side vacuum chamber 306 all independent of the main body 100, and since all the aforementioned components are integrated on the breastshield 300, there is no need to align the diaphragm 307 when arranging the breastshield 300 to the main body 100. By virtue of that the chamber cover 305 is provided with the first pipe joint 304 in communication with the air-side vacuum chamber 306, the breastshield installation chamber of the main body 100 is provided with the second pipe joint 109 in communication with the air path of the air pump 103, and the second pipe joint 109 is detachably connected to the first pipe joint 304, when the breastshield 300 is mounted to the main body 100, the air path connection of the diaphragm 307 can be completed by inserting the first pipe joint 304 with the second pipe joint 109, thereby making the cleaning and maintenance of the breastshield 300 more convenient.

Reference is made to FIGS. 4-8. In one embodiment, the base 301 of the breastshield 300 is provided with a third pipe joint 312 for outputting milk, the main body 100 is provided with a one-way valve 110 corresponding to the third pipe joint 312, and the third pipe joint 312 is detachably connected to the inlet port of the one-way valve 110. The detachable connection may be that the third pipe joint 312 is inserted into the inlet port of the one-way valve 110, or the inlet port of the one-way valve 110 is inserted into the third pipe joint 312.

In the present embodiment, the third pipe joint 312 not only serves as the milk channel 313, but also serves as a connection and positioning mechanism between the breastshield 300 and the main body 100. The use of the third pipe joint 312 simplifies the connection mechanism between the breastshield 300 and the main body 100. In addition, after the one-way valve 110 is provided, the milk in the milk collection container 200 can be prevented from flowing back.

In one embodiment, the base 301 of the breastshield 300 has a columnar main body 311, and the third pipe joint 312 extends from a side of the columnar main body 311. The breastshield installation chamber of the main body 100 has a first chamber 106, a second chamber 107 and a third chamber 108. The first chamber 106 fits with the cover body 302 and is roughly bowl-shaped. The third chamber 108 fits with the outer contour of the diaphragm installation chamber 303, and the second chamber 107 fits with the outer contour of the diaphragm installation chamber 303. The second chamber 107 is columnar and fits with the columnar chamber 311 of the base 301. In the present disclosure, the second chamber 107 is also referred to as a columnar connection chamber, the side of the columnar connection chamber is provided with an installation hole for the one-way valve 110, the columnar main body 311 is inserted into the columnar connection chamber, and is detachably connected with the columnar connection chamber.

In the present embodiment, the columnar main body 311, the columnar connection chamber, the third pipe joint 312 and the inlet port of the one-way valve 110 together form the connection and positioning mechanism between the breastshield 300 and the main body 100. When the breastshield 300 is installed on the main body 100, the third pipe joint 312 and the inlet port of the one-way valve 100 are aligned and inserted, and the columnar main body 311 is pushed into the columnar connection chamber in the main body 100 to complete the installation. When the breastshield 300 is disassembled from the main body 100, it only needs to pull the breastshield 300 outward from the main body 100, so the disassembly and installation of the breastshield 100 becomes more convenient.

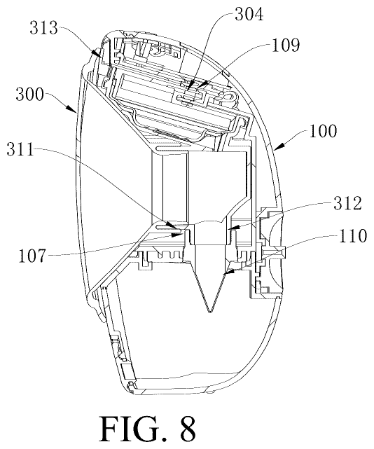

Reference is made to FIG. 8. In the present embodiment, the side of the columnar main body 311 opposite to the third pipe joint 312, more specifically, the chamber cover of the diaphragm installation chamber is provided with a convex portion 315 for the user's fingers to move. The convex portion 315 extends to the mouth edge of the breastshield 300. After the convex portion 315 is provided, the finger can easily hold the convex portion 315, so that the detachment operation of the breastshield 300 becomes easier. The convex portion may also be provided not on the chamber cover of the diaphragm installation chamber, but on other positions of the columnar main body 311.

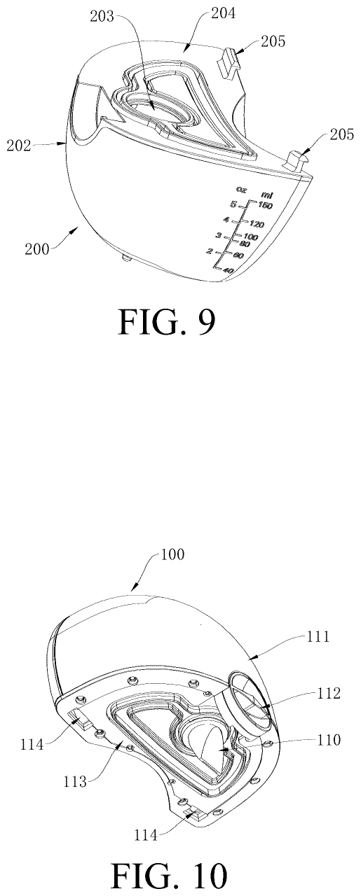

Reference is made to FIGS. 9-12, in which the main body 100 has a first end 113 for connecting to the milk collection container 200, and a second end 111 facing away from the user's breast, the first end 113 and the second end 111 are adjacent to each other. The milk collection container 200 has a third end 204 fitting with the first end 113, and a fourth end 202 forming an outer end of the breast pump together with the second end 111.

In one embodiment, the third end 204 is provided with at least one convex hook 205 extending toward the first end 113, and the first end 113 is provided with a button hole 114 fitting with the at least one convex hook 205. The convex hook 205 has a rod portion 2052 and a reverse hook portion 2051 at an end of the rod portion 2051. The hole buckle 114 has a hole 1141 through which the rod portion 2052 passes and an edge 1142 engaged with the reverse hook portion 2051, and the second end 111 is provided with a rotary lock 112 configured to lock the fourth end 202 on the second end 111.

In the present embodiment, when combining the milk collection container 200 with the main body 100, the convex hook 205 is inserted into the hole buckle 114, and then the rotary lock 112 is rotated by 90 degrees to disassemble the milk collection container 200 from the main body 100. When the main body 100 is disassembled, the rotary lock 112 is rotated by 90 degrees in the opposite direction, and then the convex hook 205 is released from the hole 114. The combination and disassembly of the milk collection container 200 and the main body 100 are very convenient.

In one embodiment, the third end 204 has an insertion hole 203 for inserting the one-way valve 110 into the milk collection container 200, and the one-way valve 110 is fixed to the first end 113 of the main body 100. In the present embodiment, the one-way valve 110 is fixed on the main body 100, so that after the milk collection container 200 is disassembled from the main body 100, the milk in the milk collection container 200 can be poured out through the insertion hole 203.

In other embodiments, the one-way valve 110 can be fixed on the milk collection container 200.

In one embodiment, a milk extraction hole is provided at a position of the milk collection container 200 away from the third end 204, and the milk extraction hole is provided with a sealing cover 201 (see FIG. 2). In this embodiment, when the sealing cover 201 is opened, the milk in the milk collection container 200 can flow out from the milk extraction hole due to gravity, so that the milk can also be taken out without disassembling the milk collection container 200, which further facilitates the operation of extracting milk from the breast pump.

The present disclosure has been described in detail through specific embodiments above. These detailed descriptions are only limited to help those skilled in the art understand the content of the present disclosure, and should not be construed as limiting the protection scope of the present disclosure. Various modifications and equivalent transformations made by those skilled in the art to the above scheme under the concept of the present disclosure shall be included in the protection scope of the present disclosure.

What is claimed is:

1. A breast pump wearable in a bra, comprising:
a main body including a battery, an air pump and a circuit control device;
a milk collection container detachably connected to the main body;
a breastshield detachably connected to the main body and in communication with the milk collection container; and
a diaphragm,
wherein the breastshield has a breast accommodating chamber, a nipple accommodating chamber and a diaphragm installation chamber; a mouth of the diaphragm installation chamber is provided with a chamber cover, the diaphragm is sealingly combined with the mouth of the diaphragm installation chamber, a milk-side vacuum chamber is formed between the diaphragm and the diaphragm installation chamber, an air-side vacuum chamber is formed between the diaphragm and the chamber cover, the chamber cover is provided with a first pipe joint in spatial communication with the air-side vacuum chamber, the diaphragm installation chamber is provided with an air hole in spatial communication with the milk-side vacuum chamber and the nipple accommodating chamber, the breastshield installation chamber of the main body is provided with a second pipe joint connected to an air path of the air pump, and the second pipe joint is detachably connected to the first pipe joint.

2. The breast pump wearable in a bra according to claim 1, wherein the breastshield includes a base made of a rigid material and a cover body made of a soft rubber, the diaphragm installation chamber is arranged on the base, the breast accommodating chamber and the nipple accommodating chamber are correspondingly arranged on the cover body, the base is connected to a periphery of the nipple accommodating chamber, and a milk channel from the nipple accommodating chamber to the milk collection container is arranged inside the base.

3. The breast pump wearable in a bra according to claim 2, wherein, the base of the breastshield is provided with a third pipe joint for outputting milk, the main body is provided with a one-way valve corresponding to the third pipe joint, and the third pipe joint is detachably connected to an inlet port of the one-way valve.

4. The breast pump wearable in a bra according to claim 3, wherein the base of the breastshield has a columnar main body, the third pipe joint extends from a side of the columnar main body, the breastshield installation chamber of the main body has a columnar connection chamber, an installation hole for the one-way valve is provided on a side of the columnar connection chamber, and the columnar main body is detachably connected to the columnar connection chamber.

5. The breast pump wearable in a bra according to claim 4, wherein one side of the columnar main body opposite to the third pipe joint is provided with a convex portion configured for user's fingers to move, and the convex portion extends to a mouth edge of the breastshield.

6. The breast pump wearable in a bra according to claim 1, wherein the main body has a first end for connecting to the milk collection container and a second end facing away from a user's breast, and the first end and the second end are adjacent to each other; the milk collection container has a third end fitting with the first end, and a fourth end forming an outer end of the breast pump together with the second end; the third end is provided with at least one convex hook extending toward the first end, the first end is provided with a hole buckle fitting with the at least one convex hook; the convex hook has a rod portion and a reverse hook portion at an end of the rod portion; the hole buckle has a hole through which the rod portion passes and an edge fittingly engaged to the reverse hook portion, and the second end is provided with a rotary lock configured to lock the fourth end on the second end.

7. The breast pump wearable in a bra according to claim 6, wherein the third end has an insertion hole for inserting the one-way valve into the milk collection container, and the one-way valve is fixed to the first end of the main body.

8. The breast pump wearable in a bra according to claim 6, wherein a milk extraction hole is provided at a position of the milk collection container away from the third end, and the milk extraction hole is provided with a sealing cover.

9. The breast pump wearable in a bra according to claim 1, wherein the circuit control device includes a Bluetooth® module configured to receive an external control signal.

10. The breast pump wearable in a bra according to claim 9, wherein the breast pump includes a dedicated remote controller.
